(19) United States
(12) Patent Application Publication
(10) Pub. No.: US 2023/0298708 A1
(43) Pub. Date: Sep. 21, 2023

(54) UNSTRUCTURED TO STRUCTURED DATA PIPELINE IN A CLINICAL TRIAL VERIFICATION SYSTEM

(71) Applicant: Akyrian Systems LLC, New York, NY (US)

(72) Inventors: Edward Ikeguchi, New York, NY (US); Peter T. Jackson, Brooklyn, NY (US)

(73) Assignee: Akyrian Systems LLC, New York, NY (US)

(21) Appl. No.: 18/122,489

(22) Filed: Mar. 16, 2023

Related U.S. Application Data
(60) Provisional application No. 63/320,393, filed on Mar. 16, 2022.

(51) Int. Cl. G16H 10/20; G16H 10/60

(52) U.S. Cl. CPC G16H 10/20; G16H 10/60

(57) ABSTRACT

The present invention provides a system and method for converting unstructured data into structured data to respond to at least one question on an electronic case report form, including taking a source capture of a portion of an electronic health record (EHR), converting unstructured data on the source capture into alphanumeric data, cross referencing the alphanumeric data with a library of terms and presenting one or more users with a list of suggestions for a response, the one or more users each selecting a user response from the list of suggestions, and reviewing the one or more user responses and selecting a final response, thereby converting the unstructured data to structured data.

This application claims priority to U.S. Provisional Patent Application No. 63/320,393, filed Mar. 16, 2022, entitled “UNSTRUCTURED TO STRUCTURED DATA PIPELINE USING THE ELECTRONIC DATA DOCUMENT (EDD),” which is hereby incorporated by reference in its entirety.

The present invention provides the ability to take images of unstructured data, such as EHR data, and convert it into research-grade structured data through the use of libraries.

### BACKGROUND

U.S. Patent Publication 2021/089589 teaches an automated system for analysis and standardizing various types of input data including structured and unstructured data. Embodiments also provide generating responses to specific questions based on standardized input data.

U.S. Patent Publication 2016/217112 teaches user-initiated data recognition and conversion process for the conversion of unstructured data to structured data from a capture of an optical character recognition image with the unstructured data underneath.

U.S. Patent Publication 2020/159824 teaches an intelligent computer platform to provide a contextual response to a question. An electronic communication interface or portal is dynamically evaluated by an artificial intelligence (AI) platform and Natural language processing (NLP) is used to detect and evaluate a communication. A corresponding factoid pipeline is searched for content related to the communication and a response is ascertained, formulated, and presented to the electronic communication interface.

U.S. Pat. No. 11,164,045 teaches a system and method for providing a platform for complex image data analysis using artificial intelligence and/or machine learning algorithms. One or more subsystems allow for the capturing of user input such as eye gaze and dictation for automated generation of findings.

The prior art cited above is related to unstructured and structured data, however, none of the prior art cited above is directed to a process for converting unstructured data to structured data in a process useful for validating clinical trial data, the focus of the present invention. Furthermore, the above cited prior art do not teach library features as provided by the present invention.

Clinical trials consist of entry of information into Case Report Forms, which has traditionally been a manual process. Even if the data is stored in an Electronic Health Record (EHR) and is being/transferred to an Electronic Case Report Form (eCRF), transcription from one to the other is still required. This transcription process is a source of potential errors.

The present invention provides a system and method that overcomes these drawbacks of the prior art.

### SUMMARY OF THE INVENTION

In U.S. Pat. Nos. 10,706,958, 10,811,122, 11,562,810, and 11,562,811, which are hereby incorporated by reference in their entirety, the inventors of the present invention teach a clinical trial verification system and method that allows an investigator to create an electronic data document (EDD) in response to a clinical trial research electronic case report form (eCRF). A snippet is created by the medical professional and included as part of the EDD to respond to one or more questions on the eCRF. Instead of the medical professional tasked with entering information by transcribing information from a patient's chart to an eCRF (i.e., the act of writing information in the form of glyphs, letters, or the like, in order to provide information in response to a question), the professional can instead use digital images of source documents (SD) to provide the required information. For example, in accordance with one or more of the above incorporated patents, the professional may be asked to log into their electronic health records (EHR) (or electronic medical records (EMR)) systems and select portions of a displayed screen that include information that answers one or more questions on the eCRF and create a source capture (SC) of an image including the one or more portions of the screen (such as a JPEG file). This effectively allows the professional to map a portion of an EHR image to an eCRF question. This workflow of clinical trial data collection has the medical professional at the medical site capture screen images and subsequently create individual snippets (source captures, or SCs) for each question of the eCRF. The medical professional then preferably reviews and electronically signs the completed form, which is then preferably transmitted to the sponsor (i.e. pharmaceutical, biotech, medical device, or other clinical trial supporting company) via the clinical trial verification system. Once received by the sponsor, data management personnel may use the snippets to confirm the accuracy of the information contained in the corresponding fields in the eCRF (clinical database) by accessing the image information included in the EDD, which incorporates the image information taken directly from the medical professional's official EHR/EMR. Using this approach, there is usually no reason to refer to a Source Document (SD) in order to validate answers. This is in contrast to prior systems in which data is transcribed, and therefore may need to be reviewed manually by third party quality control personnel (monitors) and edited to correct transcription errors. In these cases, the investigator must re-review and re-sign the documents when data has been corrected. This may lead to inefficiencies.

In PCT/US2021/33900 and PCT/US2021/62095, which are hereby incorporated by reference in their entirety, the inventors of the present invention teach a clinical trial verification system and method that describes a process where the system performs OCR on the entirety of an EHR page and creates hotspots' or suggested areas where answers to questions might exist. These hotspots' effectively allow the data on the image to be mapped to questions on the eCRF. Assisted snippet creation by “hotspots” uses “hotspots” to facilitate the process of snippet creation by providing the ability for the system to perform a computer vision technique/image analysis, such as pixel comparison, to snippets that had been submitted in the past as corresponding to an area of interest on the EHR screen capture. This is after training a dataset for machine learning, which assumes that the first time around, the process is manual, and requires an initial snippet associated with a eCRF question. For all subsequent instances where the exact same question is asked, the software would be able to generate hotspots' on the page with pre-delineated areas, with a facility for the user to accept or reject (and reposition) the markings that defined the limits of the desired snippet. Another means disclosed for facilitating snippet creation and the delineation of hotspots' includes the use of optical character recognition (OCR) to ‘read’ the text of the source capture and find key words. Any areas with key words on the EHR page would be presented to the user to accept or reject. Further disclosed is a step where the system may automatically create snippets in lieu of the hotspots based upon keyword searching. Also disclosed is a step where the system allows the user to select a system generated suggestion for a response to an eCRF question whereby the suggestion consists of an alphanumeric conversion of the snippet image into text or a menu of several possible choices based on an AI driven matching to a hierarchical list of items.

The present invention addresses a related issue, the process of converting unstructured data, which is text or other information not already associated with a type of information, from an EHR/EMR or other unstructured data source, to structured data. Long text narratives are an intrinsic part of medical record gathering during a patient encounter. Examples could include but not limited to patient history, operative reports, radiology interpretations, pathology readings and hospital notes by a clinician describing events. These are all considered to be unstructured data and are not optimized for analysis. Also considered to be unstructured data are images related to radiology, pathology, patient video or electrocardiograms where interpretation is needed to arrive at a final result that can be codified for purposes of analysis. The present invention converts this unstructured data to structured data allowing the data to be optimized for analysis.

The present invention provides a system and method for converting unstructured data into structured data to respond to at least one question on an electronic case report form, including taking a source capture of a portion of an electronic health record (EHR), converting unstructured data on the source capture into alphanumeric data, cross referencing the alphanumeric data with a library of terms and presenting one or more users with a list of suggestions for a response, the one or more users each selecting a user response from the list of suggestions, and reviewing the one or more user responses and selecting a final response, thereby converting the unstructured data to structured data.

The present invention also provides system and method for answering a question in a clinical trial questionnaire, including capturing a source capture of at least a portion of unstructured data in an electronic health record, extracting the at least a portion of the unstructured data from the source capture and converting the at least a portion of unstructured data into alphanumeric data, cross referencing the alphanumeric data with a library of terms, selecting a response from the library of terms by one or more users, and reviewing and selecting a final response by an adjudicator, wherein the adjudicator selects a final response from the one or more user responses to determine a preferred correlation between the alphanumeric data and the question in the clinical trial questionnaire.

The present invention also provides a system and method for converting unstructured data into structured data to respond to at least one question on an electronic case report form, including a source capture of a portion of an electronic health record (EHR), a converter that extracts unstructured data on the source capture into alphanumeric data, a library of terms wherein the alphanumeric data is cross referenced against the library of terms to provide one or more users with a list of suggestions for a response, a response to the question on an electronic case report form selected by the one or more users from the list of suggestions, and a final response, wherein the final response is selected from the one or more user responses.

The present invention also provides a system and method for answering a question in a clinical trial questionnaire, including a source capture of at least a portion of unstructured data an electronic health record, a converter that extracts alphanumeric data from the source capture, a library of terms, wherein the alphanumeric data is cross referenced with the library of terms, one or more users, wherein the one or more users selects a response from the library of terms, and an adjudicator, wherein the adjudicator selects a final response from the one or more user responses to determine a preferred correlation between the alphanumeric data and the question in the clinical trial questionnaire.

### BRIEF DESCRIPTION OF THE DRAWINGS

Further objects, features and advantages of the invention will become apparent from the following detailed description taken in conjunction with the accompanying figure showing an illustrative embodiment of the invention, in which:

FIG. 1 shows a flowchart of an exemplary embodiment of the present invention;

FIG. 2 shows a flowchart of an exemplary embodiment of the adjudication process; and

FIG. 3 shows a flowchart of an exemplary embodiment of a video capture of a screen.

### DETAILED DESCRIPTION OF THE INVENTION

The present invention provides the ability for a clinical trial verification system or other source of unstructured data to convert unstructured data to structured data. This process may include the following: the system may create a suggestion of a snippet and enabling the user to accept or reject the suggested area of the snippet. The system may also OCR the text of the snippet and cross reference the text of the snippet against a pre-established library of terms to categorize each term into a structured taxonomy. The user may be enabled to accept, or modify the system generated categorization or manually choose from a structured hierarchical list which is also known as a ‘Library’. The Library can be made searchable and based on the text of the snippet, the system can automatically make response suggestions for the user. The system may also utilize artificial intelligence (AI) to learn the correct associations between one or more library terms and items recurring in the unstructured data presented to the system so as to enhance its accuracy over time. The system may also take the original EDD (SC, snippet, OCR term) and add associated dimensions to the original EDD including a structured term and AI algorithms such that the EDD may be duplicated on multiple instances across other eCRF forms or other clinical trials. The added dimensions may include information such as the name of the hospital or clinic and their EHR manufacturer, so that the system can apply the same machine learning algorithms to future eCRFs in different studies using the same EHR sites. This leads to consistent mapping of data on EHR pages from the same manufacturer. For example, while many hospitals use a wide variety of electronic medical record systems, any one particular hospital, clinic or doctor's office will likely have the same EHR software for many years. The inventive system can “learn” the appearance of a particular EHR software as it is implemented for a particular medical practice and save the parameters in its database as dimensions, or metadata of the EDD. This allows faster and more accurate mapping of images from that particular medical practice's EHR if similar eCRF questions need to be answered in the future. The first time an investigator, such as a nurse, needs to supply the patient's blood pressure, they will submit a snippet. This image information will serve to train the inventive system's computer vision. For subsequent instances of the same question at the same medical practice, the same EHR software with its user interface, will be in effect. The inventive system will therefore find the correct areas for automated snippet creation in the same place each time and make suggestions with greater accuracy for the user who is responsible for creating snippets.

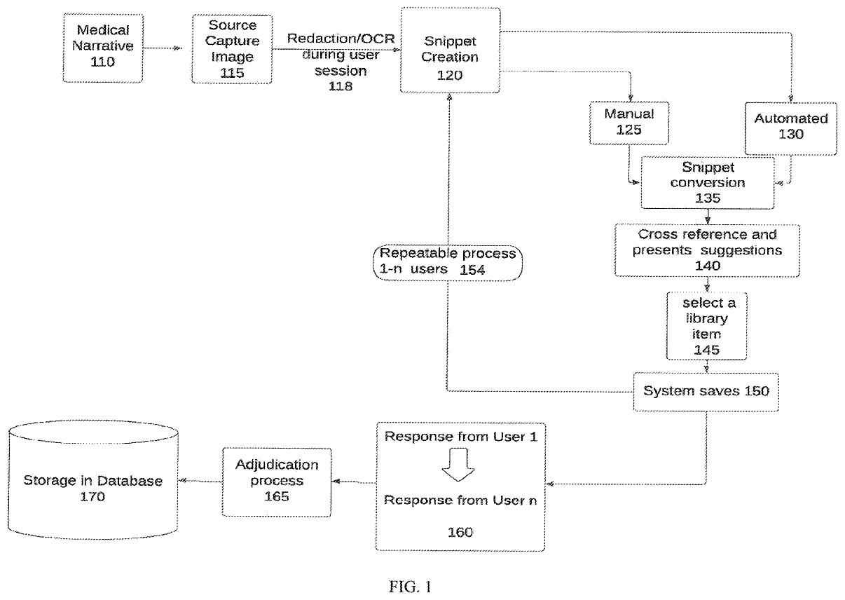

FIG. 1 shows an exemplary embodiment of a user session using the present invention. A medical narrative 110 is collected by an investigator, such as a doctor, which consists of text, images and other information from an EHR system that is recorded using screen capture, scanning, uploading of pre-existing media such as pdf files or video, or some other imaging process. The narrative mainly consists of unstructured data input by the investigator such as medical narrative, surgical narrative, x-ray pictures, endoscopy video, and/or EKG printout. A portion or all of the narrative information is then captured in one or more source captures (SC) 115. Once the SC is created, the system performs OCR on the entire source image and personally identifiable information (PII) 118 may be redacted. This occurs either automatically by the system searching the OCR content of the image for the patient's name or date of birth which have been entered by the user previously and/or manually by the user who may be provided a ‘redaction tool’ which allows them to click and drag a square area around information that they do not want to be seen by others. In the preferred embodiment, the information which is redacted by either the automated or manual process is permanently destroyed and appears blacked-out on the source image. In an alternative embodiment, the system may be configured to allow redacted areas of the image to be saved for viewing by particular users, such as auditors or regulatory personnel. In this case, the areas of redaction may have their original information retained in either image or alphanumeric form in an encrypted state. Viewing of saved redactions may require additional password entry, electronic signature, or biometric scan. Record of all sessions where content was unlocked and viewed in such fashion may be saved in an audit trail with timestamps and information about the accessing user. Once the PII is redacted, one or more snippets are created 120 to associate areas of the SC with one or more specific questions on an eCRF. The snippet may be created automatically 130 or manually 125 using a snippet crop tool. In the case of automatically generated snippets, the system preferably searches the contents of the source image and creates a snippet around a circumscribed area of interest. The user can preferably make adjustments to the system generated snippet by clicking and dragging the snippet with their mouse or resizing the snippet using handles that are present in the snippet's corners. The snippet image is then converted to alphanumeric data based on the previously performed OCR 135 where the system determines which OCR text corresponds to the area of the image within the snippet. The alphanumeric text data converted from the snippet is then used to cross reference a library of items that have been codified, such as lists of medications, (for example WHODrug), medication diagnoses (for example, MedDra or ICD-10), and the like, and present the user with a list of suggested response options 140. The list of suggested options may be driven by natural language processing or other artificial intelligence (AI). For example, initial suggestions may be chosen to create a most likely candidates list of options for the user. After the user continues to select the same choice a few times, the AI may narrow down the list of options or even auto populate the response field based on prior user behavior. For example, a reading of “120/80” may be automatically recognized as a heart rate/blood pressure reading. Additionally, “chronic knee pain” may be coded as Osteoarthritis (ICD-10 code M19.9), and thus associated by the AI-driven process. Either the user or the AI selects the relevant library item 145. The inventive system saves the original full-page source image, the original snippet, any associated text from the original snippet and the selected library term 150. This process is repeatable 155 with 1 through n users, such as clinical site coordinators, nurses, physicians, and/or third-party subject matter experts that may not be associated with the clinical trial site. Each selected library item term in each user process (1-n) is saved and considered a response 160. An adjudication process 165 takes the one or more responses 160 and selects a single final response. The adjudication process may be made by one or more users for the final selection of the structured data. During the adjudication process at least one user is designated to review each of the coding response choices against the original content in a blinded fashion where they are unaware of the names of other users. The final selection, as well as all user responses, adjudicator actions, and all metainformation, are stored in a storage database 170 in an audit trail. As the process continues, the loop for answering questions on the eCRF may eventually be AI driven in which only 1 suggestion is provided for the user to accept or reject. This is after training a dataset for machine learning, which assumes that the first time around determining responses, the process is manual, and requires an initial selection of a suggested library item. As the users continue to select a suggested response, the software would be able to generate a single suggestion, with a facility for the user to accept or reject.

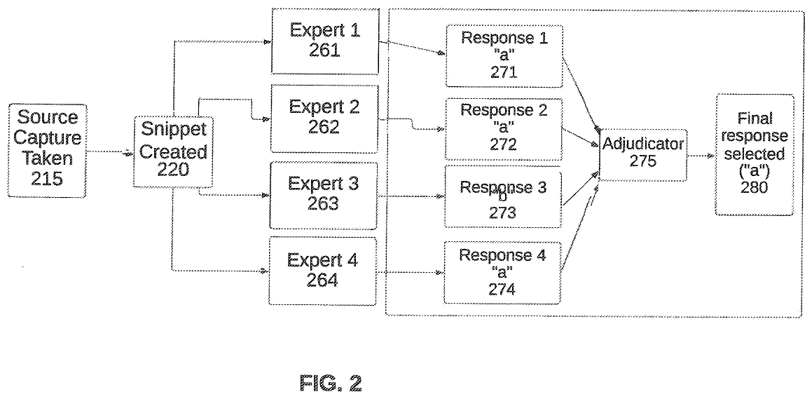

FIG. 2 shows a flowchart of an exemplary embodiment of the adjudication process in further detail. Once a source capture is taken 215 and a snippet is created 220 individual users (1-n) generate responses 261, 262, 263, 264 as discussed in detail in FIG. 1. An adjudicator views all of the responses (271, 272, 273, 274) provided 275 to determine the final response 280. The adjudicator reviews all of the responses to determine which response most accurately answers the eCRF question.

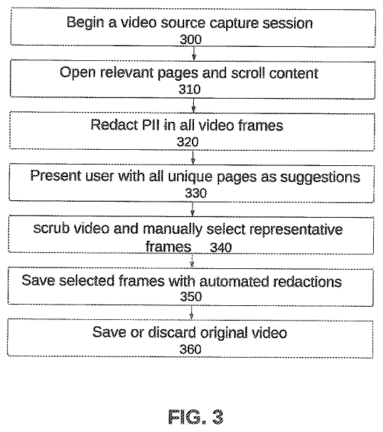

In order to facilitate source capture of multiple EHR pages to be as comprehensive of the patient's visits as possible, the system may allow the capture of multiple EHR screen images, The user is given the ability to take a single SC and after redaction, the SC is saved in a queue. After all of the multiple SC have been taken by the user and redacted, the user can return to the queue to access all of the SCs that were taken. For example, the doctor (investigator) may take multiple SCs of all the relevant EHR pages for a single patient visit. The investigator may also conduct a ‘source capture session’ where the system records a video of the user's screen, thus capturing imagery of each of the EHR pages as they were browsed, ultimately allowing the system to scrape the necessary data from the acquired media. FIG. 3 shows a flow chart for a source capture from a video source capture session. The user begins recording a video source capture session 300 and opens all of the relevant pages and scrolls through the EHR content 310. (For example, recording in a Google Meet session with screen sharing). Video is taken of the selected EHR software while the user is opening and scrolling through the pages. After the recording is completed, the system processes the video and redacts all of the PII in the each of the video frames 320. The inventive system may provide the user with unique pages as suggestions 330 driven by AI or the system provides the user with an interface in which the user may manually scrub forwards and backwards through the video and ‘freeze’/select any frames that are representative of a SC to be used to answer to an eCRF question. by the user and the user may manually select representative frames for a SC 340. All selected frames are saved with automated redactions 350 and the original video or only selected frames may be saved or discarded 360 based on the system configuration. All of the videos/frames are encrypted and redacted with inputs into the audit trail before passing outputs to the snippeting process and other downstream functions.

Furthermore, in order to add greater depth to the ability to convert unstructured to structured data, the present invention may take on several embodiments to understand the underlying page elements of the EHR system from which the SC was taken and the relationships between those page elements by using a process in computer science known as introspection. This includes where the system may introspect elements of a web based EHR system using the browser's document object model (DOM) API; and/or where the system may introspect elements of a ‘heavy client’ (i.e., desktop installation) EHR system using Windows Forms API or other relevant Windows UI APIs. This would allow the system to identify where on a page a response was located, for example, in a ‘header’ or ‘footer’ or in a particular division or so called ‘<div>’ in HTML.

General

The present invention can take the form of an entirely hardware embodiment, an entirely software embodiment, or an embodiment containing both hardware and software elements. In an exemplary embodiment, the present invention is implemented in software, which includes but is not limited to firmware, resident software, and microcode. Any conventional EMR system can be used as a source of SC, such as Cerner, Epic Systems and Allscripts. The upload of standalone files such as JPEGs, multipage PDF documents or images of paper documents may also be used. These go through the same redaction and OCR process. Preferably, the systems and methods of the present invention are implemented in a client/server network connected via the Internet.

The systems and methods of the present invention will also preferably use data security, encryption, and data capture and transfer protocols that will enhance patient privacy and security and add desirable authentication and verification features. Preferably, all data will be transferred over SSL connections (also known as HTTPS). Preferably, the systems and methods of the invention will use data encryption (public/private key pair) to protect patient medical records represented in SD media, and data encryption will protect both data and media while they are stored and while they are being transferred, ensuring that only the intended recipients are able to access/view them. Preferably, every user must be authenticated on the system by logging in with their private credentials. Preferably, during each interaction with the server, the server confirms the authenticity of the request for interaction by authentication tokens issued by the server. Preferably, the system will require the users to change their passwords periodically. Preferably, users will only have access to the functionality assigned to them by the system administrator. Preferably, information such as patient or subject ID, data capture date, and other necessary identifying information is embedded in the SC and SD image itself as well as included in metadata, and accessible to qualified users and viewers of the EDD. This may also include a unique identifying serial number, subject ID, date/time of capture, IP addresses, user information such as user web browser and device type, for example.

In another preferred embodiment, no media or data is saved to any local machine or device, either by the machine or device as it is created or by the monitor/Sponsor or Investigator when viewing it. Rather, data is captured directly from the screen output by the inventive software and is not handled by the native Operating System, which might write that data to disk, even if only as temporarily cached files. Any additional image processing that may be required, such as file compression for storage, is handled by servers away from the local machine or device. Preferably, the computer systems and programs used in embodiments of the invention do not save the EDD or other files created incident to the operation of the invention as a file that can be recalled at a later time. SC can be obtained from any EMR software running on the same machine as the inventive software, such that the EMR software displays its information on the same screen(s) as are accessible by software implementing the invention. Additional digital media imported by the invention from any external source, such as photographs of paper documents or medical scans, are treated in the same manner as SC once loaded.

Furthermore, the present invention can take the form of a computer program product or products accessible from a computer-usable or computer-readable medium providing program code for use by or in connection with a computer system or any instruction execution system. The computer program product includes the instructions that implement the method of the present invention. A computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk, and an optical disk. Current examples of optical disks include compact disk-read only memory (CD-ROM), compact disk-read/write (CD-R/W), and DVD.

A computer system suitable for storing and/or executing program code includes at least one processor coupled directly or indirectly to memory elements through a system bus. The memory elements include local memory employed during actual execution of the program code, bulk storage, and cache memories that provide temporary storage of at least some program code to reduce the number of times code is retrieved from bulk storage during execution. Input/output (I/O) devices (including but not limited to keyboards, displays, pointing devices, etc.) can be coupled to the computer system either directly or through intervening I/O controllers. Network adapters may also be coupled to the computer system in order to enable the computer system to become coupled to other computer systems or remote printers or storage devices through intervening private or public networks. Modems, cable modems, and Ethernet cards are just a few of the currently available types of network adapters. The computer system can also include an operating system and a computer filesystem.

It is to be understood that the above description and examples are intended to be illustrative and not restrictive. Many embodiments will be apparent to those of skill in the art upon reading the above description and examples. The scope of the invention should, therefore, be determined not with reference to the above description and examples but should instead be determined with reference to the appended claims, along with the full scope of equivalents to which such claims are entitled. The disclosures of all articles and references, including patent applications and publications, are incorporated herein by reference for all purposes.

1. A method for converting unstructured data into structured data to respond to at least one question on an electronic case report form, the method comprising:
taking a source capture of a portion of an electronic health record (EHR);
converting unstructured data on the source capture into alphanumeric data;
cross referencing the alphanumeric data with a library of terms and presenting one or more users with a list of suggestions for a response;
the one or more users each selecting a user response from the list of suggestions; and
reviewing the one or more user responses and selecting a final response, thereby converting the unstructured data to structured data.

2. The method as recited in claim 1, further comprising storing some or all of the final response, the one or more user responses and associated metainformation in a storage database.

3. The method as recited in claim 1, wherein the list of suggestions is AI driven.

4. The method as recited in claim 1, further comprising saving the structured data and the library term.

5. A system for converting unstructured data into structured data to respond to at least one question on an electronic case report form, the system comprising:
a source capture of a portion of an electronic health record (EHR);
a converter that extracts unstructured data on the source capture into alphanumeric data;
a library of terms wherein the alphanumeric data is cross referenced against the library of terms to provide one or more users with a list of suggestions for a response;
a response to the question on an electronic case report form selected by the one or more users from the list of suggestions; and
a final response, wherein the final response is selected from the one or more user responses.

6. The system as recited in claim 5, further comprising a storage database storing some or all of the final response, the one or more user responses and associated metainformation.

7. The system as recited in claim 5, wherein the list of suggestions is AI driven.

8. The system as recited in claim 5, further comprising a storage database for storing the alphanumeric data and the library term.

9. A method for answering a question in a clinical trial questionnaire, the method comprising
capturing a source capture of at least a portion of unstructured data in an electronic health record;
extracting the at least a portion of the unstructured data from the source capture and converting the at least a portion of unstructured data into alphanumeric data;
cross referencing the alphanumeric data with a library of terms;
selecting a response from the library of terms by one or more users; and
reviewing and selecting a final response by an adjudicator, wherein the adjudicator selects a final response from the one or more user responses to determine a preferred correlation between the alphanumeric data and the question in the clinical trial questionnaire.

10. The method as recited in claim 9, further comprising redacting personally identifiable information from the alphanumeric data.

11. The method as recited in claim 9, wherein selecting a response from the library of terms is AI driven.

12. The method as recited in claim 9, wherein the final response selected by the adjudicator is further used by an AI driven process as an input for selecting the response from the library of terms by the one or more users for subsequent source captures.

13. A system for answering a question in a clinical trial questionnaire, the system comprising
a source capture of at least a portion of unstructured data an electronic health record;
a converter that extracts alphanumeric data from the source capture;
a library of terms, wherein the alphanumeric data is cross referenced with the library of terms;
one or more users, wherein the one or more users selects a response from the library of terms; and
an adjudicator, wherein the adjudicator selects a final response from the one or more user responses to determine a preferred correlation between the alphanumeric data and the question in the clinical trial questionnaire.

14. The system as recited in claim 13, further comprising a redactor component for redacting personally identifiable information from the alphanumeric data.

15. The system as recited in claim 13, wherein the final response selected by the adjudicator is further used by an AI driven process as an input for selecting the response from the library of terms by the one or more users for subsequent source captures.
